(19) United States
(12) Patent Application Publication
(10) Pub. No.: US 2025/0325845 A1
(43) Pub. Date: Oct. 23, 2025

(54) ADJUSTABLE PROBE FOR TRANSCRANIAL ULTRASOUND

(71) Applicant: Sanmai Technologies, PBC, Sunnyvale, CA (US)

(72) Inventors: Joseph SANGUINETTI, Tucson, AZ (US); Quentin Owen TODD, Foster City, CA (US); Stephanie FRENCH, Sunnyvale, CA (US); Jon Christopher LAKE, Sunnyvale, CA (US); Weijun WANG, Menlo Park, CA (US); Christopher DAFT, Tucson, AZ (US)

(21) Appl. No.: 19/170,935

(22) Filed: Apr. 4, 2025

Related U.S. Application Data
(60) Provisional application No. 63/637,810, filed on Apr. 23, 2024.

(51) Int. Cl. A61N 7/02; A61N 7/00

(52) U.S. Cl. CPC A61N 7/02; A61N 2007/0026

(57) ABSTRACT

A cranial probe assembly comprises an ultrasound transducer packaged into a probe. A stable probe holder in the form of a fixed ring is positioned on a patient's head. The position of the probe relative to the patient's head is adjustable with five degrees of freedom comprising cartesian coordinates x and y, the z-axis normal to the probe, and two orientation angles orthogonal to the z-axis. The adjustments assure the efficacy of an ultrasound procedure.

### CROSS-REFERENCE TO RELATED APPLICATIONS

This application claims priority benefit to U.S. Provisional Patent Application No. 63/637,810, filed Apr. 23, 2024 and entitled “ADJUSTABLE PROBE FOR TRANSCRANIAL ULTRASOUND”, the contents of which are incorporated by reference herein in its entirety.

### TECHNICAL FIELD

This invention relates to a medical ultrasound system that employs a probe that has an adjustable position and orientation on a patient's head.

### BACKGROUND

To achieve efficacy with a medical ultrasound system it is important to provide a stable mounting of the ultrasound equipment on a patient's head. Furthermore, it is necessary to adjust the position and orientation of a temporal probe so that the stimulation beam it produces is properly focused on a target anatomy. In an ultrasound system that lacks the capability to steer its ultrasound beam in response to unintended motion of the probe, stability of the probe's location with respect to the head is critically important. Probe motion either laterally across the head, or as an angular displacement, can be destructive to the effectiveness of the treatment. A high level of probe stability is required for clinical success.

Second, experience has shown that patients are sensitive to structures in contact with the head. This constrains the type of headset which can support a transducer during transcranial ultrasound treatments. For example, patients may be sensitive to the weight of the headset and its probe, how it rests on their skin, the materials which comprise the areas of patient contact, and other factors.

Third, it is difficult to reduce the weight of a traditional bulk PZT probe below approximately 100 g, which complicates the headset design. This weight constraint is a consequence of several factors. The diameter of the probe needs to be large enough, for example 70 mm, to adequately focus ultrasound at a frequency low enough to penetrate the skull without large attenuation or beam aberration. The thickness of the piezoelectric material is constrained by its resonant frequency to, for example, around 4 mm for a 500 kHz resonance. Since the density of typical lead zirconate titanate (PZT) is around 7.6 kg/cm³, small material volumes result in significant weight. Matching layers, an acoustic lens and the probe case add to the overall weight. Epoxy backing material coating the back side of the transducer stack can be quite thick and is typically doped with tungsten, resulting in a high material density.

Human subjects are always in motion, even if the patient is asked to restrict their movements during the procedure. The length of the procedure is determined by medical needs but can easily be 10-20 minutes, or more. Therefore, a need exists for improved headset designs.

### SUMMARY OF THE INVENTION

The headset described herein provides stability of the ultrasonic probe during a clinical session, creates a comfortable human interface, and is light in weight while providing an effective probe diameter. For clinical efficacy, the position of the probe relative to the position of the patient's head can be adjusted with five degrees of freedom. The five degrees of freedom comprise cartesian coordinates x and y, axis z normal to the probe, and two orientation angles orthogonal to the z-axis.

### BRIEF DESCRIPTION OF THE DRAWINGS

FIG. 1 illustrates a prior art ultrasound treatment system.

FIG. 2 illustrates a headset, in accordance with an embodiment of the present disclosure.

FIG. 3 illustrates a stable mounting system for an ultrasonic probe, in accordance with an embodiment of the present disclosure.

FIG. 4 illustrates a mechanism for adjusting the x-y coordinates of an ultrasonic probe, in accordance with an embodiment of the present disclosure.

FIG. 5 illustrates a cross-sectional view of a probe assembly mated with a fixed ring, including a screw for adjusting the spacing and pressure of an ultrasonic probe on a patient's head, in accordance with an embodiment of the present disclosure.

FIG. 6 illustrates a hydrogel pad to be provided with an ultrasonic probe, for a comfortable fit on a patient's head, in accordance with an embodiment of the present disclosure.

FIG. 7 illustrates an acoustic coupling consumable which fits inside the device, in accordance with an embodiment of the present disclosure.

FIG. 8 illustrates a fiducial adapter with rotational capability around two axes, in accordance with an embodiment of the present disclosure.

### DETAILED DESCRIPTION OF THE INVENTION

Attaching an ultrasound probe to a patient's head for transcranial neuromodulation is difficult for several reasons. Embodiments of the disclosure address various drawbacks present in conventional solutions. The setup time just before the ultrasonic stimulation is started is critical for the system operator and the patient. The headset must be able to be put on easily, and once it is in place, the probe must be moved to the correct treatment location and fixed in place. Once fixed, it should press against the head with enough force to exclude air from the path of the ultrasound as it travels from the probe to the scalp. However, the force must not be large enough to create discomfort for the patient. Examples of the disclosure provide a headset capable of providing various geometric features associated with ultrasonic stimulation, while maintaining comfort for the patient.

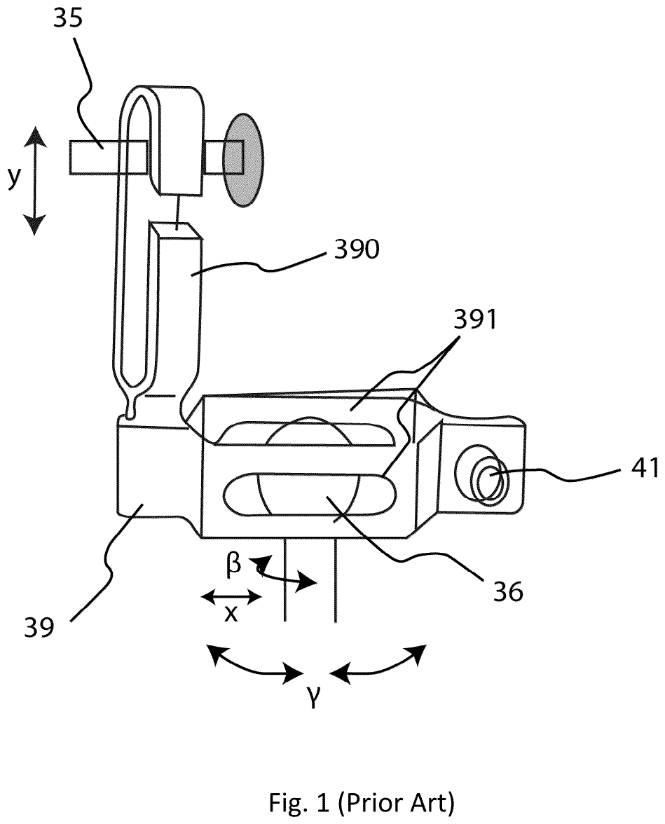

FIG. 1 illustrates a prior art ultrasound treatment system described in prior art patent Ultrasound treatment system and ultrasonic diagnostic system, JP200639504A, issued to Makoto Ogiwara et al. The ultrasonic treatment system is provided with an ultrasonic probe holding means for holding an ultrasonic probe, and a movement fixing means which holds the ultrasonic probe holding means and which three-dimensionally moves and fixes the ultrasonic probe holding means to a position to be irradiated of a patient. The ultrasonic probe holding means is provided with a means for rotating the ultrasonic probe with the axial direction of the ultrasonic probe as a center. The ultrasonic probe holding means is provided with a spring mechanism for pushing the ultrasonic probe out to the side of the patient. The movement fixing means is provided with a means for linearly moving the ultrasonic probe holding means; and a means for rotate-moving the ultrasonic probe holding means. As shown in the figure, the adjuster arm 39 is provided with a vertically long opening 390 so that the adjuster arm 39 can move up and down in the y-axis direction. A screw is passed through the opening 390 and the adjuster arm 39 is fixed to a temporal ultrasonic probe fixing unit using the screw 35. The adjuster arm 39 is sandwiched and fixed between the head of screw 35 and the temporal ultrasonic fixing unit. The movement in the z-axis direction is realized by sliding the screw 35, and at the same time, the movement in the y-axis direction is realized by moving the adjuster arm 39. The screw 35 can be constructed from nylon in one embodiment.

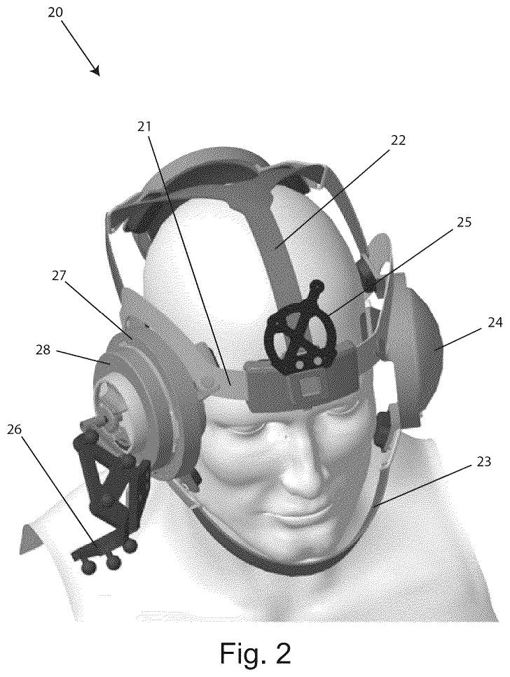

FIG. 2 illustrates a headset 20 attached to a patient's head, comprising a forehead band 21, an overhead strap 22, a chinstrap 23, and a counterweight 24. Fiducials are used as reference points, enabling accurate correlation between an ultrasound or MRI modality and the patient's physical anatomy. They facilitate the alignment of a pre-operative MRI scan with real-time images obtained during the ultrasound procedure. This “registration” ensures that surgeons or clinicians have a clear, up-to-date view of the anatomical structures they are working with. The fiducials help in guiding the probe to specific target areas such as targets of radiation therapy. A first fiducial 25 and a second fiducial 26 are shown. These appear as distinct points in acquired images, allowing for their identification and localization. Headset 20 provides a secure mounting of fixed ring 27 to the patient's head, which attaches to probe holder 28 and is compatible with many probe adjustments described herein.

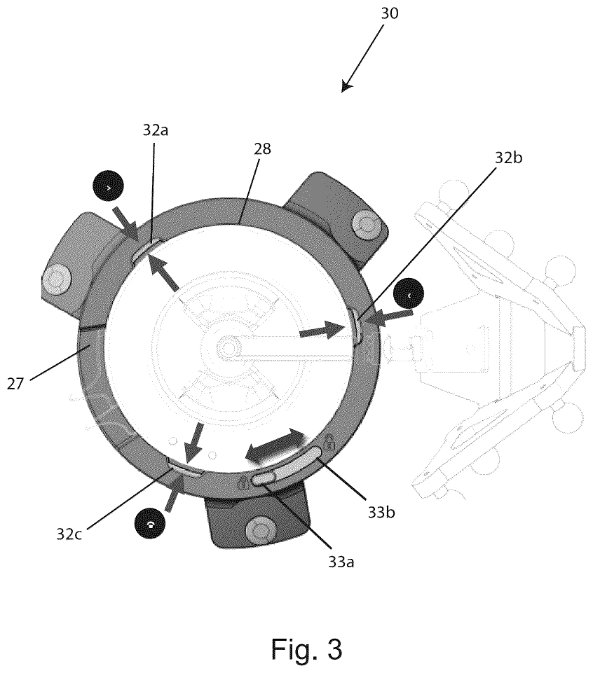

FIG. 3 illustrates configuration 30 for mating probe holder 28 with fixed ring 27. In one embodiment, the probe holder 28 includes three feet that engage with corresponding recesses or notches in fixed ring 27 at three symmetrically located positions 32a, 32b, and 32c. When the clinician has moved the probe to the desired position, based on feedback from the navigation system, slider 33a is moved to the locked position as shown; unlocked position 33b is also shown.

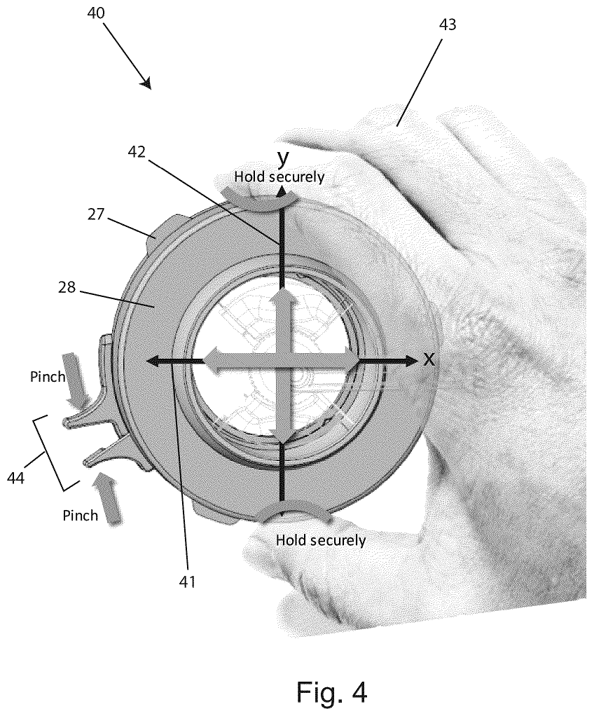

FIG. 4 illustrates a translation assembly 40 for moving the probe holder 28 with respect to the fixed ring 27. A sliding action allows the probe to move in an x-direction or a y-direction or both, as shown by the horizontal and vertical arrows in FIG. 4. In some embodiments, the translation provided is approximately 10 millimeters in at least one of the x and y directions. Pinching the pinch trigger 44 releases probe holder 28 held by a clutch mechanism to move with respect to fixed ring 27. The clutch mechanism comprises: (i) a clutch backing element; (ii) an outer ring of elastomeric clutch material; (iii) a clutch pressure plate that allows three fingers positioned above the outer ring to engage with the elastomeric material when a pinch trigger is released; (iv) a ramp mechanism coupled to the pinch trigger to separate the fingers from the elastomeric material, allowing the probe to move up to 10 mm in an x or direction with respect to the holding ring. An operator's hand 43 is shown firmly gripping probe holder 28 for moving it in a sliding fashion when pinch trigger 44 is pinched.

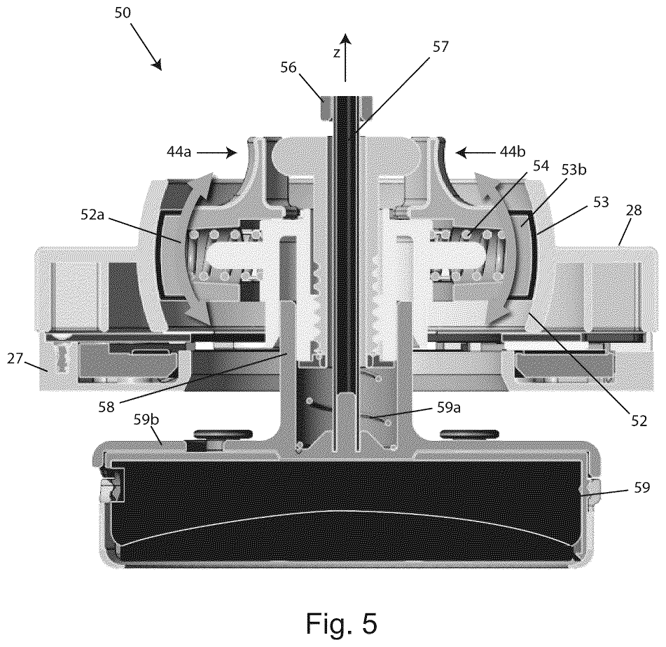

FIG. 5 illustrates a cross-sectional view 50 of a probe holder 28 mated with fixed ring 27 and including additional features. Fixed ring 27 is coupled via housing 53b to probe holder 28 using a ball joint 52. A brake is formed at the ball-joint surface using a compressible material 53 that is pressed against the ball joint surface using springs 54. In an embodiment, material 53 comprises rubber. By unpinching trigger 44, a braking action is enabled. Accordingly, the braking action comprises engagement of compressible material 53 backed by springs 54 with a spherical surface 52 inside the probe holder 28.

The braking effect can be canceled by pinching on the pinch trigger 44. Ball joint 52 enables angular adjustment in two dimensions orthogonal to the z-axis, one of which, θ, is shown by arrow 52a. The other angular dimension φ is not shown but is orthogonal to arrow 52a. The size of ball joint 62 and the length of compressible material 53 combine to provide 10° of angular adjustment in each of these angular dimensions. A screw 56 is provided for adjusting the pressure provided to a patient's skin in the z-axis, screw 56 operating on the gap closure slider 58 connected to probe case 59b; screw 56 adjusts the separation distance between them. Screw 56 is working against spring 59a. Screw 56 can be constructed of nylon in some embodiments. Shaft 57 of a fiducial assembly is shown, aligned with the z-axis of the center of the ultrasound probe holder 28.

Five degrees of freedom have been described: cartesian coordinates x and y which are adjusted as described in reference to FIG. 4; z-axis normal to the probe which is described in reference to FIG. 5; and two orientation angles θ and φ, orthogonal to the z-axis, also described in reference to FIG. 5.

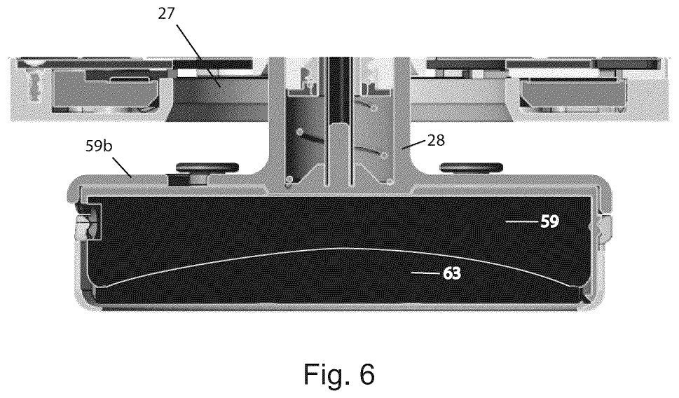

FIG. 6 illustrates the probe case 59b comprising the piezoelectric stack 59 to which is coupled to z a cylindrical semi-solid gel layer 63 or a compressible material. Layer 63 may also be described as a hydrogel pad. This arrangement provides a comfortable interface with a patient's skin.

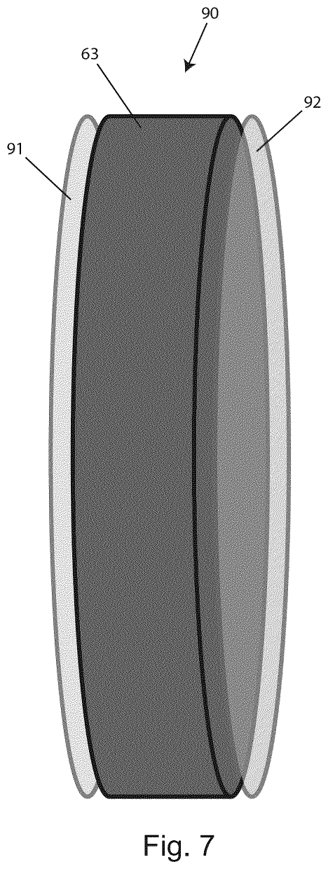

FIG. 7 illustrates an improvement 90 on the design of FIG. 6 for the coupling component of cylinder 63. The coupling component is inserted between the probe and the head of the patient. This component meets several requirements. It is soft and compliant with the contour of the subject's head, to exclude air in the ultrasonic path, which would reduce the treatment efficacy. Also, it is desirable for the surface touching the skin and the surface touching the probe to be gently adhesive with a tape layer to further minimize the risk of movement of parts during the treatment. The adhesive for the skin side needs to be biocompatible while the adhesive for the probe side needs to bond well (but temporarily) to the surface of the probe, which may be comprised of silicone. These adhesives may not be compatible with the hydrogel (or other semi-solid gel) 63. Therefore, it is advantageous for this component to be made in three pieces as shown in FIG. 7. Note that in FIG. 7, the three parts are spaced slightly apart for clarity of illustration. In practice, tapes 91 and 92 are glued to gel 63. Item 91 is a tape, ideally made of a soft polymer, which is very thin compared to the acoustic wavelength. For example, its thickness may be 25-50 microns. It has a biocompatible adhesive on its left-hand face in the FIG. 7, and an adhesive specialized for the gel 63 on its right-hand face. Item 92 is a similarly thin polymer tape with gel-compatible adhesive on its left-hand face and adhesive specialized for the probe surface on its right-hand face.

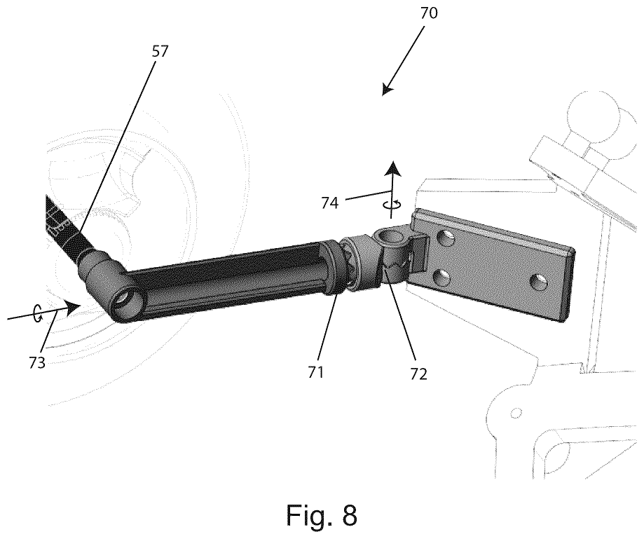

FIG. 8 illustrates a fiducial adapter 70 with rotational capability around two axes. Shaft 57 of the fiducial adapter is shown. Fiducial adapter 70 comprises two joints, 71 and 72, where each joint is capable of 360° rotation. Joint 71 provides 360° rotation about axis 73, and joint 72 provides 360° rotation about axis 74.

1. In some embodiments, a cranial probe assembly comprises an ultrasound transducer packaged in a probe, and a probe holder positioned on a patient's head, wherein the position of the probe relative to the position of the patient's head can be adjusted with five degrees of freedom.

2. The cranial probe assembly of clause 1, wherein the probe attaches to a fixed ring using a plurality of symmetrically located feet, and wherein the attachment comprises a lock and an unlock position.

3. The cranial probe assembly of clauses 1 or 2, wherein the five degrees of freedom comprise cartesian coordinates x and y, axis z normal to the probe, and two orientation angles orthogonal to axis-z.

4. The cranial probe assembly of any of clauses 1-3, wherein adjustment with respect to cartesian coordinates x and y is accomplished using a pinch trigger to release an assembly comprising a ring of elastomeric material that acts as a brake when engaged with opposing fingers that are coupled to the probe

5. The cranial probe assembly of any of clauses 1-4, wherein the brake assembly employs pads comprising a compressible material.

6. The cranial probe assembly of any of clauses 1-5, wherein the compressible material comprises silicone rubber.

7. The cranial probe assembly of any of clauses 1-6, wherein adjustment of x and y comprises a translation of up to approximately 10 millimeters in at least one of the x and y directions.

8. The cranial probe assembly of any of clauses 1-7, wherein a ball joint is provided between the probe holder and the probe case, wherein pinching a pair of clamps while rotating the probe holder within the housing enables angular adjustment.

9. The cranial probe assembly of any of clauses 1-8, wherein the adjustment of the angular orientation is performed in response to rotation of the probe holder.

10. The cranial probe assembly of any of clauses 1-9, wherein each of the two orientation angles has an adjustment range of approximately 10 degrees.

11. The cranial probe assembly of any of clauses 1-10, wherein releasing a pinch trigger enables a braking action.

12. The cranial probe assembly of any of clauses 1-11, wherein the braking action comprises engagement of compressible material with a spherical surface of the housing, wherein the compressible material is backed by a spring.

13. The cranial probe assembly of any of clauses 1-12, wherein adjustment with respect to the z-axis comprises rotation of a screw located at the z-axis.

14. The cranial probe assembly of any of clauses 1-13, wherein the screw is rotatable using an operator's fingers.

15. The cranial probe assembly of any of clauses 1-14, wherein the screw comprises nylon.

16. The cranial probe assembly of any of clauses 1-15, wherein the separation distance between the probe and a patient's head can be adjusted using the screw.

17. The cranial probe assembly of any of clauses 1-16, further comprising a coupling component inserted between the probe and the head of a patient, wherein the coupling component comprises a semi-solid gel.

18. The cranial probe assembly of any of clauses 1-17, wherein the coupling component comprises a semi-solid gel layer and at least one adhesive tape layer.

19. The cranial probe assembly of any of clauses 1-18 further comprising a fiducial assembly having a shaft that is inserted along the z axis of the probe.

20. The cranial probe assembly of any of clauses 1-19 wherein the fiducial assembly further comprises an adapter that can be rotated 360° along each of two axes.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method, or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A cranial probe assembly comprising:
an ultrasound transducer packaged in a probe; and
a probe holder positioned on a patient's head, wherein the position of the probe relative to the position of the patient's head can be adjusted with five degrees of freedom.

2. The cranial probe assembly of claim 1, wherein the probe attaches to a fixed ring using a plurality of symmetrically located feet, and wherein the attachment comprises a lock and an unlock position.

3. The cranial probe assembly of claim 1, wherein the five degrees of freedom comprise cartesian coordinates x and y, axis z normal to the probe, and two orientation angles orthogonal to axis-z.

4. The cranial probe assembly of claim 3, wherein adjustment with respect to cartesian coordinates x and y is accomplished using a pinch trigger to release an assembly comprising a ring of elastomeric material that acts as a brake when engaged with opposing fingers that are coupled to the probe.

5. The cranial probe assembly of claim 4, wherein the brake assembly employs pads comprising a compressible material.

6. The cranial probe assembly of claim 5, wherein the compressible material comprises silicone rubber.

7. The cranial probe assembly of claim 3, wherein adjustment of x and y comprises a translation of up to approximately 10 millimeters in at least one of the x and y directions.

8. The cranial probe assembly of claim 3, wherein a ball joint is provided between the probe holder and the probe case, wherein pinching a pair of clamps while rotating the probe holder within the housing enables angular adjustment.

9. The cranial probe assembly of claim 8, wherein the adjustment of the angular orientation is performed in response to rotation of the probe holder.

10. The cranial probe assembly of claim 3, wherein each of the two orientation angles has an adjustment range of approximately 10 degrees.

11. The cranial probe assembly of claim 8, wherein releasing a pinch trigger enables a braking action.

12. The cranial probe assembly of claim 11, wherein the braking action comprises engagement of compressible material with a spherical surface of the housing, wherein the compressible material is backed by a spring.

13. The cranial probe assembly of claim 3, wherein adjustment with respect to the z-axis comprises rotation of a screw located at the z-axis.

14. The cranial probe assembly of claim 13, wherein the screw is rotatable using an operator's fingers.

15. The cranial probe assembly of claim 13, wherein the screw comprises nylon.

16. The cranial probe assembly of claim 13, wherein the separation distance between the probe and a patient's head can be adjusted using the screw.

17. The cranial probe assembly of claim 1, further comprising a coupling component inserted between the probe and the head of a patient, wherein the coupling component comprises a semi-solid gel.

18. The cranial probe assembly of claim 17, wherein the coupling component comprises a semi-solid gel layer and at least one adhesive tape layer.

19. The cranial probe assembly of claim 3 further comprising a fiducial assembly having a shaft that is inserted along the z axis of the probe.

20. The cranial probe assembly of claim 19 wherein the fiducial assembly further comprises an adapter that can be rotated 360° along each of two axes.
